SEQUENCE LISTING (32 sequences)

SEQ ID NO: 1 - DNA, 18 bp, Artificial Sequence
  inventor Uematsu, Chihiro ; Gouda Chifumi; Anazawa Takashi; Shirai Masataka
aagtgacctttaaacatg

SEQ ID NO: 2 - DNA, 18 bp, Artificial Sequence
  Description of Artificial Sequence FITC labeled probe unit
aagtgagctttaaaccag

SEQ ID NO: 3 - DNA, 18 bp, Artificial Sequence
  Description of Artificial Sequence FITC labeled probe unit
aagtggtctttaaacgcg

SEQ ID NO: 4 - DNA, 18 bp, Artificial Sequence
  Description of Artificial Sequence FITC labeled probe unit
aagtgtactttaaactgg

SEQ ID NO: 5 - DNA, 18 bp, Artificial Sequence
  Description of Artificial Sequence FITC labeled probe unit
aagtgatctttaaacacg

SEQ ID NO: 6 - DNA, 18 bp, Artificial Sequence
  Description of Artificial Sequence FITC labeled probe unit
acttgacctttccacatg

SEQ ID NO: 7 - DNA, 18 bp, Artificial Sequence
  Description of Artificial Sequence FITC labeled probe unit
acttgagctttccaccag

SEQ ID NO: 8 - DNA, 18 bp, Artificial Sequence
  Description of Artificial Sequence FITC labeled probe unit
acttggtctttccacgcg

SEQ ID NO: 9 - DNA, 18 bp, Artificial Sequence
  Description of Artificial Sequence FITC labeled probe unit
acttgtactttccactgg

SEQ ID NO: 10 - DNA, 18 bp, Artificial Sequence
  Description of Artificial Sequence FITC labeled probe unit
acttgatctttccacacg

SEQ ID NO: 11 - DNA, 16 bp, Artificial Sequence
  Description of Artificial Sequence oligonucleotide having complementary sequences of probe units
ggtcacttgcacattt

SEQ ID NO: 12 - DNA, 17 bp, Artificial Sequence
  Description of Artificial Sequence oligonucleotide having complementary sequencess of probe units
gctcacttcatgtttaa

SEQ ID NO: 13 - DNA, 17 bp, Artificial Sequence
  Description of Artificial Sequence oligonucleotide having complementary sequences of probe units
gaccacttctggtttaa

SEQ ID NO: 14 - DNA, 17 bp, Artificial Sequence
  Description of Artificial Sequence oligonucleotide having complementary sequences of probe units
gtacacttcgcgtttaa

SEQ ID NO: 15 - DNA, 17 bp, Artificial Sequence
  Description of Artificial Sequence oligonucleotide having complementary sequences of probe units
gatcacttccagtttaa

SEQ ID NO: 16 - DNA, 17 bp, Artificial Sequence
  Description of Artificial Sequence oligonucleotide having complementary sequences of probe units
ggtcaagtcgtgtttaa

SEQ ID NO: 17 - DNA, 17 bp, Artificial Sequence
  Description of Artificial Sequence oligonucleotide having complementary sequences of probe units
gctcaagtcatgtggaa

SEQ ID NO: 18 - DNA, 17 bp, Artificial Sequence
  Description of Artificial Sequence oligonucleotide having complementary sequences of probe units
gaccaagtctggtggaa

SEQ ID NO: 19 - DNA, 17 bp, Artificial Sequence
  Description of Artificial Sequence oligonucleotide having complementary sequences of probe units
gtacaagtcgcgtggaa

SEQ ID NO: 20 - DNA, 17 bp, Artificial Sequence
  Description of Artificial Sequence oligonucleotide having complementary sequences of probe units
gatcaagtccagtggaa

SEQ ID NO: 21 - DNA, 31 bp, Artificial Sequence
  Description of Artificial Sequence probe unit which hybridizes to target molecule
ctagccgagtagtgttgggttttaaatgtgc

SEQ ID NO: 22 - DNA, 16 bp, Artificial Sequence
  Description of Artificial Sequence biotin labeled probe unit
gatgagctttggacca

SEQ ID NO: 23 - DNA, 16 bp, Artificial Sequence
  Description of Artificial Sequence biotin labeled probe unit
gatggtctttggacgc

SEQ ID NO: 24 - DNA, 16 bp, Artificial Sequence
  Description of Artificial Sequence biotin labeled probe unit
gatgtactttggactg

SEQ ID NO: 25 - DNA, 16 bp, Artificial Sequence
  Description of Artificial Sequence biotin labeled probe unit
gatgatctttggacac

SEQ ID NO: 26 - DNA, 16 bp, Artificial Sequence
  Description of Artificial Sequence biotin labeled probe unit
tctgacctttttacat

SEQ ID NO: 27 - DNA, 16 bp, Artificial Sequence
  Description of Artificial Sequence oligonucleotide having complementary sequences of probe units
gctcatctcatgtcca

SEQ ID NO: 28 - DNA, 16 bp, Artificial Sequence
  Description of Artificial Sequence oligonucleotide having complementary sequences of probe units
gaccatctctggtcca

SEQ ID NO: 29 - DNA, 16 bp, Artificial Sequence
  Description of Artificial Sequence oligonucleotide having complementary sequences of probe units
gtacatctcgcgtcca

SEQ ID NO: 30 - DNA, 16 bp, Artificial Sequence
  Description of Artificial Sequence oligonucleotide having complementary sequences of probe units
gatcatctccagtcca

SEQ ID NO: 31 - DNA, 16 bp, Artificial Sequence
  Description of Artificial Sequence oligonucleotide having complementary sequences of probe units
ggtcagatcgtgtcca

SEQ ID NO: 32 - DNA, 31 bp, Artificial Sequence
  Description of Artificial Sequence unit probe which hybridizes to target molecule
ctagccgagtagtgttgggttttatggacat

(19) United States
(12) Patent Application Publication
(10) Pub. No.: US 2007/0238125 A1
(43) Pub. Date: Oct. 11, 2007

(54) Probe synthesis method for nucleic acid detection

(76) Inventors: Chihiro Uematsu, Kawasaki (JP); Chifumi Gouda, Kokubunji (JP); Masataka Shirai, Higashimurayama (JP); Takashi Anazawa, Koganei (JP)

Correspondence Address: REED SMITH LLP;Suite 1400, 3110 Fairview Park Drive, Falls Church, VA, 22042, US

(21) Appl. No.: 11/783,124

(22) Filed: Apr. 6, 2007

(30) Foreign Application Priority Data
Apr. 10, 2006 (JP) 2006-107876

(51) Int. Cl. C12Q 1/68; C12P 19/34; C12M 3/00

(52) U.S. Cl. 435/6; 435/91.2; 435/287.2; 427/2.11

(57) ABSTRACT

The present invention relates to a probe synthesis method and a probe synthesis kit for nucleic acid detection, which are intended for the fluorophore labeling and detection of a target gene. In this method, a probe unit for analyte nucleic acid recognition having a base sequence specific to an analyte nucleic acid and plural labeled probe units each having a fluorophore labeled internal base are hybridized to oligonucleotides complementary thereto, and the adjacent probe units are bonded by ligation. As a result, an analytical probe with increased fluorescence intensity is synthesized easily and inexpensively, while the number of fluorophore labels is controlled.

### CLAIM OF PRIORITY

The present application claims priority from Japanese application JP 2006-107876 filed on Apr. 10, 2006, the content of which is hereby incorporated by reference into this application.

### BACKGROUND OF THE INVENTION

1. Field of the Invention

The present invention relates to probe synthesis for particular gene detection. More specifically, the present invention relates to a probe synthesis method and a probe synthesis kit for nucleic acid detection, which are intended for the fluorophore labeling and detection of a target gene.

2. Background Art

The analysis of gene expression levels and protein abundance with high sensitivity and high precision in a wide dynamic range plays an exceedingly important role in gene or protein functional analysis and in disease research or diagnosis. For example, infectious disease diagnosis requires quantifying infectious virus genes in the early stage for avoiding infection spread and effectively treating the infectious disease. Cancer diagnosis is conducted by measuring the serum concentration of a protein known as a cancer marker. Alternatively, pharmaceutical fields require quantifying gene expression levels or protein abundance varying specifically to disease for identifying a target in drug development or discovery and evaluating medicinal effects.

A real-time PCR method (Klein et al., Electrophoresis, 1999, 20, 291-299) has been known as a method of examination of gene expression levels. In the real-time PCR method, analyte genes present in a standard sample and in a test sample are separately subjected to PCR amplification in different reaction containers, and their amplification efficiencies are compared to thereby indirectly quantify the analyte gene. In this method, the amplified analyte gene is labeled with an intercalator that binds to double-stranded DNA and emits fluorescence, or alternatively, the analyte gene is labeled with a molecular beacon (S. Tyagi, F. R. Kramer, Nature Biotechnology, 1996, 14, 303-308) or TaqMan probe (Pamela M., et al., Proc. Natl. Acad. Sci., USA, August 1991, 88, 7276-7280) that specifically hybridizes to the analyte gene during amplification and emits fluorescence. Then, the analyte gene is quantified by measuring the amplitude of fluorescence intensity in the reaction container. Specifically, the analyte gene present in the test sample is not counted on a one molecule-by-one molecule basis but is quantified on the basis of the total amount of amplification products present in the reaction container after amplification. Examples of gene amplification methods other than PCR include NASBA (J. Compton, et. al., Nature, 1991, 350, 91-92) and TMA (JP Patent No. 3241717) methods.

On the other hand, protein abundance is often quantified by capturing a target protein by use of labeled antibodies and, after bind/free separation, measuring the amount of the labeled antibodies on the basis of fluorescence or chemiluminescence. In this case as well, the analyte protein is not counted on a one molecule-by-one molecule basis but is quantified on the basis of fluorescence intensity or luminescence intensity in the reaction solution.

Probes often used in gene and protein detection are simple oligonucleotide probes labeled with a single fluorophore, in addition to the intercalator, molecular beacon, and TaqMan probe. The molecular beacon or TaqMan probe is a probe comprising an oligonucleotide strand labeled with two fluorophores (or with one fluorophore and one quencher). These probes permit for homogeneous detection by utilizing the fluorescent resonance energy transfer between plural fluorophores. Alternatively, in multicolor fluorescence detection, ET primers (Jingyue Ju, et al., Nucleic Acids Res., 1996, 24, 1144-1148) have also been known which utilize fluorescent resonance energy transfer to eliminate a different in excitation efficiency between fluorophores. However, the ET primers do not provide order-of-magnitude increases in fluorescence intensity as compared with the simple probes, because substantially one fluorophore participates in light emission. Therefore, these probes cannot be used for the purpose of quantification by the measurement of the fluorescence intensity of one probe molecule and is used for measuring the fluorescence intensity of the whole reaction container, as described above.

By contrast, a branched probe (U.S. Pat. No. 5,124,246) has been developed as a probe intended to increase the fluorescence intensity of one probe molecule. In this probe, a linker is introduced around the halfway position of the oligonucleotide strand, and the branch portions of the oligonucleotide branched in a branch or comb form are respectively labeled with fluorophores or with enzymes for chemiluminescence to thereby increase fluorescence intensity or luminescence intensity obtained from one probe molecule.

From these viewpoints, the fluorescence detection of one probe molecule by use of a conventional detection probe required measuring fluorescence by use of an exceedingly small measurement volume in such a way that, for example, only an evanescent irradiation (Funatsu et al., Nature., 1995, 374, 555-559) region is measured. As a result, its detection limit concentration was inevitably approximately 10000 times higher than a detection limit concentration (approximately 10⁻¹⁸ M) in real-time PCR. Therefore, a general method for practical examination of gene expression levels involved amplifying an analyte gene, then measuring the total amount of the amplification products by use of a detection probe, and estimating therefrom the amount of the analyte gene present before amplification. The use of the branched probe permits for the quantification of the analyte gene without amplifying it. However, branched probe synthesis itself is generally difficult. Besides, the fluorophore labeling of each of the branched portions is also difficult. Therefore, the branched probe cannot achieve easy and inexpensive quantification.

Another possible method comprises increasing a measurement volume by use of single-molecule measurement by side irradiation and thereby lowering a detection limit concentration and achieving high sensitivity (Anazawa et al., Anal. Chem, 2002, 74, 5033-5038). However, increases in measurement volume cause reductions in laser power density and rises in background. Therefore, only the labeling of an analyte gene with one fluorophore cannot achieve detection.

An object of the present invention is to provide an easy and inexpensive synthesis method of an analytical probe with increased fluorescence intensity.

### SUMMARY OF THE INVENTION

The present inventors have found an inexpensive and easy synthesis method of a probe labeled with plural fluorophores by linking oligonucleotides each labeled with one fluorophore by ligation reaction. Specifically, plural kinds of oligonucleotides each having a fluorophore labeled base that is located internal except at the 5′-end and the 3′-end are prepared and hybridized to complementary oligonucleotides to thereby form double-stranded oligonucleotides, followed by linking by ligation to thereby synthesize one probe labeled with plural fluorophores. In this method, the fluorophore labeled oligonucleotides and an oligonucleotide having a sequence for analyte nucleic acid recognition are linked to thereby synthesize a probe having the sequence for analyte nucleic acid recognition and plural fluorophore labels. The fluorophore labeled oligonucleotides are designed as sequences irrelevant to the sequence for analyte nucleic acid recognition. As a result, the probe is synthesized so that it hybridizes to an analyte nucleic acid in a 1:1 relationship.

Specifically, the present invention provides an analytical probe production method comprising: a first step wherein a probe unit for analyte nucleic acid recognition having a base sequence specific to an analyte nucleic acid and plural labeled probe units having mutually different base sequences and each being labeled with a fluorophore are hybridized to plural complementary oligonucleotides each having a sequence complementary to a portion of the neighborhood of the 5′-end of one of the probe units and to a portion of the neighborhood of the 3′-end of one of the other probe units to thereby obtain a hybridized probe unit comprising the probe unit for analyte nucleic acid recognition, the plural labeled probe units, and the plural complementary oligonucleotides; and a second step wherein the adjacent probe units in the hybridized probe unit are bonded by ligation to thereby obtain an analytical probe labeled with plural fluorophores.

When gaps are present between the probe units in the hybridized probe unit, DNA polymerase and substrate dNTP are allowed to act on the hybridized probe unit before the ligation to thereby fill these gaps.

In the method, it is desired for increasing ligation efficiency that the fluorophore should be bound with a base located five or more bases internal from the 5′-end and 3′-end of the labeled probe unit. Moreover, it is desired that the fluorophore should be bound with a base located substantially at the center of the labeled probe unit. It is also preferred that the labeled probe unit should have a length between 9 nucleotides and 30 nucleotides inclusive.

The plural labeled probe units may be labeled with the fluorophores that are mutually identical or may be labeled with the fluorophores that are mutually different.

The present invention also provides an analytical probe production method comprising: a first step wherein a probe unit for analyte nucleic acid recognition having a base sequence specific to an analyte nucleic acid and plural probe units to be labeled having mutually different base sequences are hybridized to plural complementary oligonucleotides each having a sequence complementary to a portion of the neighborhood of the 5′-end of one of the probe units and to a portion of the neighborhood of the 3′-end of one of the other probe units to thereby obtain a hybridized probe unit comprising the probe unit for analyte nucleic acid recognition, the plural probe units to be labeled, and the plural complementary oligonucleotides; a second step wherein the adjacent probe units in the hybridized probe unit are bonded by ligation; and a third step wherein a fluorophore is introduced into each of the plural probe units to be labeled to thereby obtain an analytical probe labeled with plural fluorophores.

When gaps are present between the probe units in the hybridized probe unit, DNA polymerase and substrate dNTP are allowed to act on the hybridized probe unit before the ligation to thereby fill these gaps.

In the method, it is desired for increasing ligation efficiency that the fluorophore should be introduced into a base located five or more bases internal from the 5′-end and 3′-end of the probe unit to be labeled. Moreover, it is desired that the fluorophore should be introduced into a base located substantially at the center of the probe unit to be labeled. It is also preferred that the probe unit to be labeled should have a length between 9 nucleotides and 30 nucleotides inclusive.

The fluorophores that are mutually identical or the fluorophores that are mutually different may be introduced into the plural probe units to be labeled.

In the analytical probe production methods of the present invention, the probe unit for analyte nucleic acid recognition may be a branched probe having branches (U.S. Pat. No. 5,124,246).

The sequences of the probe units are designed to cause only predetermined reaction to proceed during ligation reaction and prevent the generation of a concatemer comprising the same sequences linked repetitively. As a result, the number of fluorophores introduced into a probe can be controlled.

The present invention also provides a kit for analytical probe production comprising: plural probe units having mutually different base sequences; and plural complementary oligonucleotides each having a sequence complementary to a portion of the neighborhood of the 5′-end of one of the probe units and to a portion of the neighborhood of the 3′-end of one of the other probe units.

In one embodiment, one of the plural probe units is a probe unit for analyte nucleic acid recognition having a base sequence specific to an analyte nucleic acid, and the plural probe units other than this probe unit for analyte nucleic acid recognition have a base sequence nonspecific to the analyte nucleic acid.

The plural probe units other than the probe unit for analyte nucleic acid recognition are labeled probe units each labeled with a fluorophore. Preferably, the labeled probe unit has a length between 9 nucleotides and 30 nucleotides inclusive and has the fluorophore in a base located five or more bases internal from the 5′-end and 3′-end.

The present invention further provides an analytical probe obtained by any of the afore-mentioned methods. The probe of the present invention is an analytical probe comprising: a probe unit for analyte nucleic acid recognition having a base sequence specific to an analyte nucleic acid; and tandemly ligated plural labeled probe units having mutually different base sequences and each comprising a fluorophore labeled internal base, wherein the fluorophore labeled internal bases are placed at a distance of eight or more bases.

In the present invention, the sequences of the fluorophore labeled probe units or the probe units to be labeled can be designed arbitrarily and can be used commonly to varying analyte nucleic acids. Accordingly, analytical probes for various analyte nucleic acids can be synthesized only by redesigning the sequences of the probe unit for analyte nucleic acid recognition and the complementary oligonucleotide for ligating this probe unit to the labeled probe unit or the probe unit to be labeled.

The analyte nucleic acid can be treated with plural fluorophore labels in a short time without synthesizing RCR products (Lizardi et al., Nature Genetics, 1998, 19, 225-232), by synthesizing the probe of the present invention in advance.

The present invention can conveniently and inexpensively synthesize a probe labeled with plural fluorophores by linking oligonucleotides each having a fluorophore labeled internal base by ligation. This can achieve improvement in the performance of probes used for various fluorescence detections and in detection limit. Moreover, the present invention allows for single-molecule measurement with sufficient sensitivity in a state in which a laser irradiation volume is increased by side irradiation and a measurement volume is kept large.

### BRIEF DESCRIPTION OF THE DRAWINGS

FIG. 1 is a diagram showing procedures to synthesize a probe labeled with plural fluorophores by ligation and detect a target molecule by the hybridization of the probe thereto;

FIG. 2 is a diagram showing procedures to synthesize a probe labeled with plural fluorophores by the hybridization of oligonucleotides (probe units) to complementary oligonucleotides and subsequent ligation;

FIG. 3 is a diagram showing procedures to synthesize a probe labeled with plural fluorophores by the linking of fluorophore unlabeled oligonucleotides (probe units to be labeled) by ligation and subsequent fluorophore labeling;

FIG. 4 is a diagram showing procedures to synthesize a probe labeled with plural fluorophores by the hybridization of fluorophore labeled oligonucleotides (labeled probe units) to complementary oligonucleotides, gap filling by use of DNA polymerase, and subsequent ligation;

FIG. 5 is a diagram showing procedures to synthesize a probe comprising plural fluorophores of plural kinds introduced therein by the linking of oligonucleotides (labeled probe units) labeled with various fluorophores;

FIG. 6 is a diagram showing procedures to synthesize a branched probe labeled with plural fluorophores by the ligation of oligonucleotides (labeled probe units) to the branch portions of the branched probe;

FIG. 7 is a diagram showing procedures to synthesize a probe labeled with plural fluorophores by the hybridization of fluorophore labeled oligonucleotides (labeled probe units) to complementary oligonucleotides and subsequent ligation;

FIG. 8 is a graph showing the comparison in fluorophore intensity between a probe labeled with a single fluorophore and a probe labeled with plural fluorophores;

FIG. 9 is a graph showing the relationship between the position of a fluorophore labeled base and ligation efficiency in the ligation of oligonucleotides (labeled probe units) each having a fluorophore labeled internal base;

FIG. 10 is a diagram showing procedures to synthesize a probe labeled with plural fluorophores by the hybridization of biotin labeled oligonucleotides (labeled probe units) to complementary oligonucleotides, gap filling by use of DNA polymerase, and subsequent ligation, followed by fluorophore labeling; and

FIG. 11 is a fluorescence microscope image showing the measurement of a probe labeled with one quantum dot and a probe labeled with plural quantum dots.

### DETAILED DESCRIPTION OF THE PREFERRED EMBODIMENTS

Hereinafter, the present invention will be described in detail with reference to drawings.

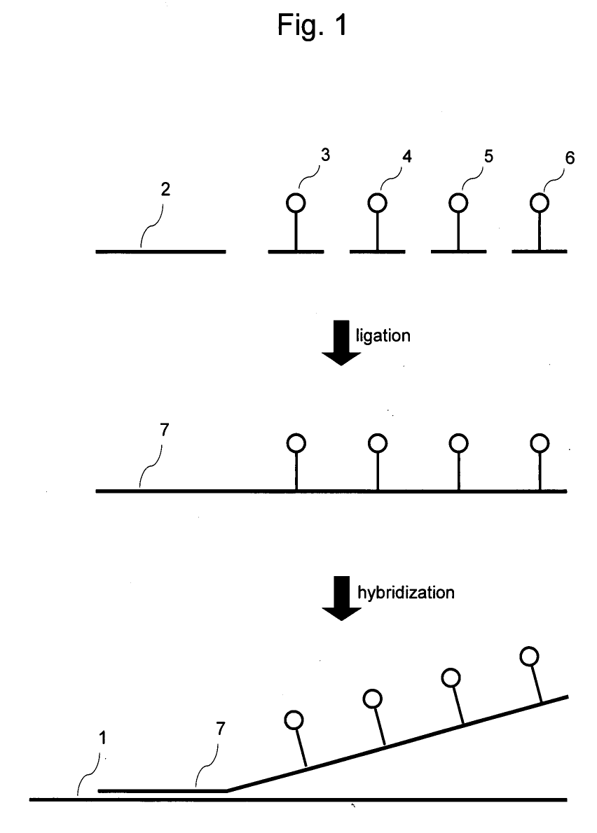

FIG. 1 is a diagram showing procedures to synthesize a fluorophore labeled probe by the present invention and detect a target molecule by the hybridization of the probe thereto. Reference numeral 1 denotes an analyte (or target) molecule (analyte nucleic acid), and reference numeral 2 denotes an oligonucleotide (probe unit for analyte nucleic acid recognition) designed to hybridize to the analyte molecule. Reference numerals 3, 4, 5, and 6 denote oligonucleotides (labeled probe units) each having a fluorophore labeled base that is located internal except at the 5′-end and the 3′-end. The oligonucleotides 2, 3, 4, 5, and 6 are linked by ligation to thereby obtain an oligonucleotide probe 7 labeled with plural fluorophores. The analyte molecule is detected by the hybridization of the oligonucleotide probe 7 thereto.

The length of an analyte molecule hybridizing portion in the sequence of the oligonucleotide is not particularly limited and is preferably approximately 18 to 25 nucleotides. This is because the existing probability of a sequence a little less than 20 nucleotides in length is one or less in a random base sequence having a length approximately equal to that of the human genome and as a result, the oligonucleotide can specifically recognize the analyte molecule. Moreover, the length of the fluorophore labeled oligonucleotide is not particularly limited and is preferably approximately 9 to 30 nucleotides.

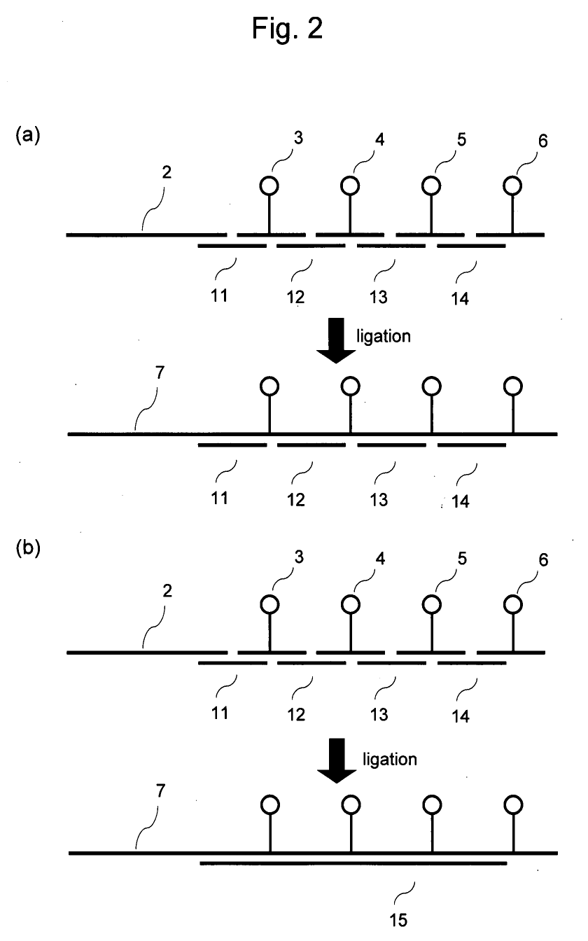

FIG. 2 is a schematic diagram specifically showing ligation reaction. An oligonucleotide (probe unit for analyte nucleic acid recognition) hybridizing to an analyte molecule and fluorophore labeled oligonucleotides (labeled probe units) are hybridized to oligonucleotides (complementary oligonucleotides) each having a sequence complementary thereto to thereby form double-stranded DNA. Then, the linking between the oligonucleotide hybridizing to an analyte molecule and the fluorophore labeled oligonucleotide adjacent thereto and between the adjacent fluorophore labeled oligonucleotides is performed by ligation to thereby synthesize an oligonucleotide probe labeled with plural fluorophores. Reference numeral 2 denotes an oligonucleotide (probe unit for analyte nucleic acid recognition) designed to hybridize to the analyte molecule. Reference numerals 3, 4, 5, and 6 denote oligonucleotides (labeled probe units) each having a fluorophore labeled base that is located internal except at the 5′-end and the 3′-end. Reference numeral 11 denotes an oligonucleotide (complementary oligonucleotide) having a sequence complementary to a portion of the sequences of the oligonucleotides 2 and 3; reference numeral 12 denotes an oligonucleotide (complementary oligonucleotide) having a sequence complementary to a portion of the sequences of the oligonucleotides 3 and 4; reference numeral 13 denotes an oligonucleotide (complementary oligonucleotide) having a sequence complementary to a portion of the sequences of the oligonucleotides 4 and 5; and reference numeral 14 denotes an oligonucleotide (complementary oligonucleotide) having a sequence complementary to a portion of the sequences of the oligonucleotides 5 and 6. The linking between the oligonucleotides 2 and 3, between the oligonucleotides 3 and 4, between the oligonucleotides 4 and 5, and between the oligonucleotides 5 and 6 is performed by ligation to thereby synthesize an oligonucleotide probe 7 labeled with plural fluorophores.

The fluorophore labeled oligonucleotides 3, 4, 5, and 6 and the complementary oligonucleotide strands 11, 12, 13, and 14 are designed so that they are hybridized only to their intended sequences to prevent mishybridization or so that their Tm values (melting temperatures) serving as an index for hybridization stability are kept as equal as possible. The ligation may be performed sequentially in such a way that the oligonucleotides 2 and 3 are linked, then, the oligonucleotide 4 is linked to the linked oligonucleotides, and further, the oligonucleotide 5 is linked thereto, or may be performed at once. Moreover, only the fluorophore labeled oligonucleotide strands may be linked as shown in FIG. 2(A), while both the fluorophore labeled oligonucleotide strands and the complementary strands may be linked as shown in FIG. 2(B).

The positions of the fluorophore labels in the fluorophore labeled oligonucleotides 3, 4, 5, and 6 are set to a position five or more bases internal from the 5′-end and the 3′-end, because the positions of the fluorophore labels within the 2nd to 4th bases from both the ends reduce ligation efficiency. This may be because the steric hindrance of the fluorophores and linkers influences enzyme reaction. Since the same tendency is observed at both the 5′-end and the 3′-end, the positions of the fluorophore labels are set to bases located internal except at both the ends to at the 4th base position therefrom. Therefore, the fluorophore labels in the fluorophore labeled oligonucleotide probe obtained after the ligation are placed at a distance of at least eight or more bases.

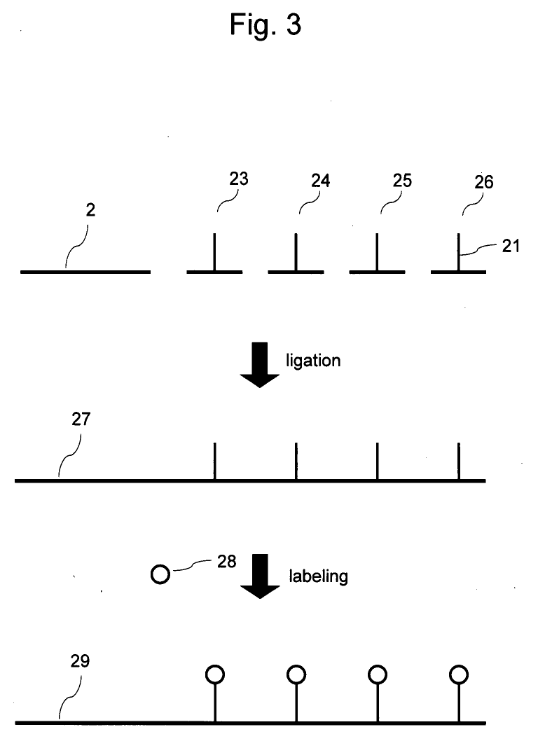

The timing of fluorophore labeling for oligonucleotides may be either before or after ligation. Fluorophore labeled oligonucleotides may be linked to thereby synthesize an oligonucleotide probe labeled with plural fluorophores, as shown in FIGS. 1 and 2, while oligonucleotides (probe units to be labeled) 23, 24, 25, and 26 each having a linker 21 for fluorophore introduction may be linked to thereby synthesize an oligonucleotide 27, followed by introduction of fluorophores 28 into the linker portions to thereby synthesize a probe 29, as shown in FIG. 3. Examples of the linkers for fluorophore introduction include, but not limited to, biotin, an amino group, and a thiol group. The position of the linker is set to a base located internal except at both the ends to at the 4th base position therefrom, as with the fluorophore.

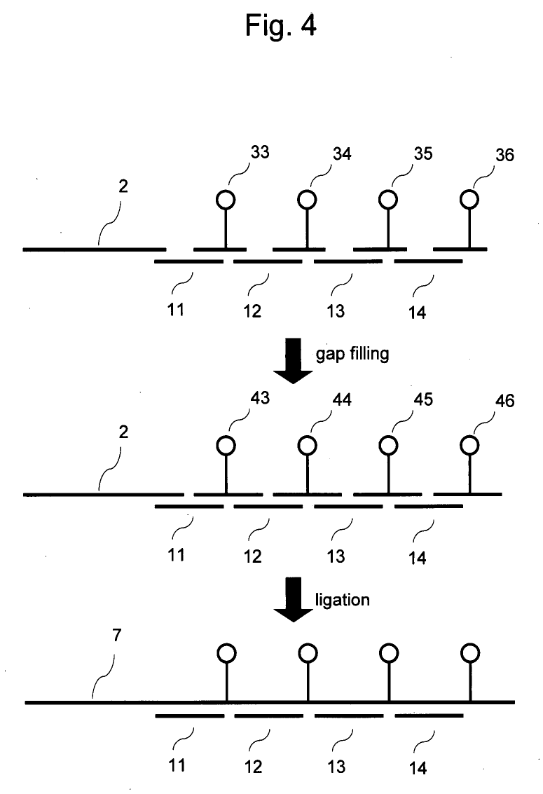

Alternatively, the fluorophore labeled oligonucleotides may be designed to produce 1-base or more gaps therebetween in their hybridization to the complementary oligonucleotides. In this case, unintended ligation products attributed to mishybridization can be prevented by gap filling by use of DNA polymerase and subsequent ligation. As shown in FIG. 4, an oligonucleotide (probe unit for analyte nucleic acid recognition) 2 hybridizing to an analyte molecule and fluorophore labeled oligonucleotides (labeled probe units) 33, 34, 35, and 36 are hybridized to oligonucleotides (complementary oligonucleotides) 11, 12, 13, and 14 complementary thereto. The sequences of the oligonucleotides 33, 34, 35, and 36 are designed to produce 1-base or more gaps therebetween in their hybridization to the oligonucleotides 11, 12, 13, and 14 complementary thereto. Next, the fluorophore labeled oligonucleotides 33, 34, 35, and 36 are elongated by use of DNA polymerase to thereby fill the gaps. As a result, they are converted to oligonucleotides 43, 44, 45, and 46. Then, ligation is performed to thereby obtain an oligonucleotide probe 7 labeled with plural fluorophores. In this case as well, the fluorophores may be introduced into linkers after the ligation:

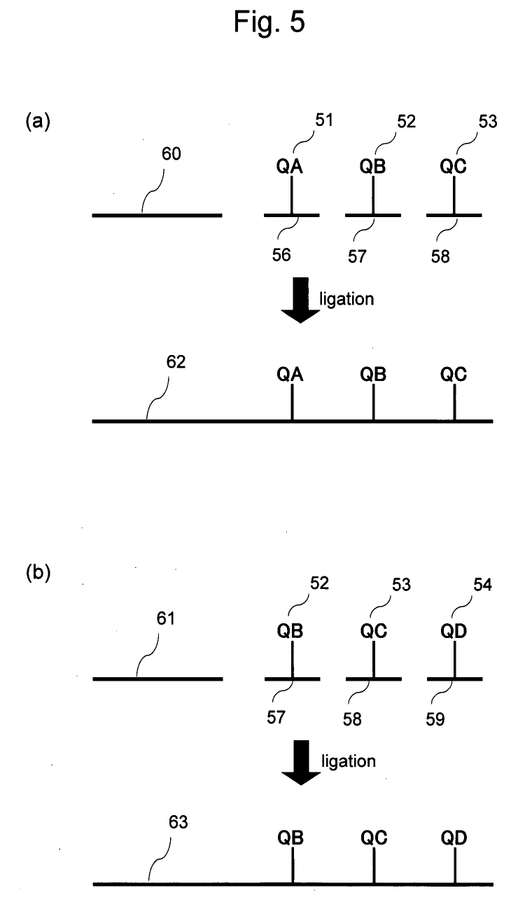

As shown in FIGS. 5(A) and 5(B), oligonucleotides (labeled probe units) 56, 57, 58, and 59 respectively labeled with various fluorophores 51, 52, 53, and 54 and oligonucleotides (probe units for analyte nucleic acid recognition) 60 and 61 recognizing mutually different analyte molecules can be prepared and linked to thereby obtain probes 62 and 63 labeled with plural fluorophores of plural kinds. The fluorescence wavelength of the whole probe can be controlled by changing the kinds of fluorophores.

Since quantum dots can be excited in a wide wavelength range, the use of the quantum dots as fluorophores can easily synthesize probes having various fluorescence wavelengths. Plural analyte molecules can be distinguished on the basis of fluorescence wavelengths and tested by one measurement by changing the sequences recognizing the analyte molecules and changing the combination of fluorophores (quantum dots) used according to the sequences. When m kinds are selected and used from n kinds of fluorophores, ₙCₘ kinds of fluorescent probes can be synthesized.

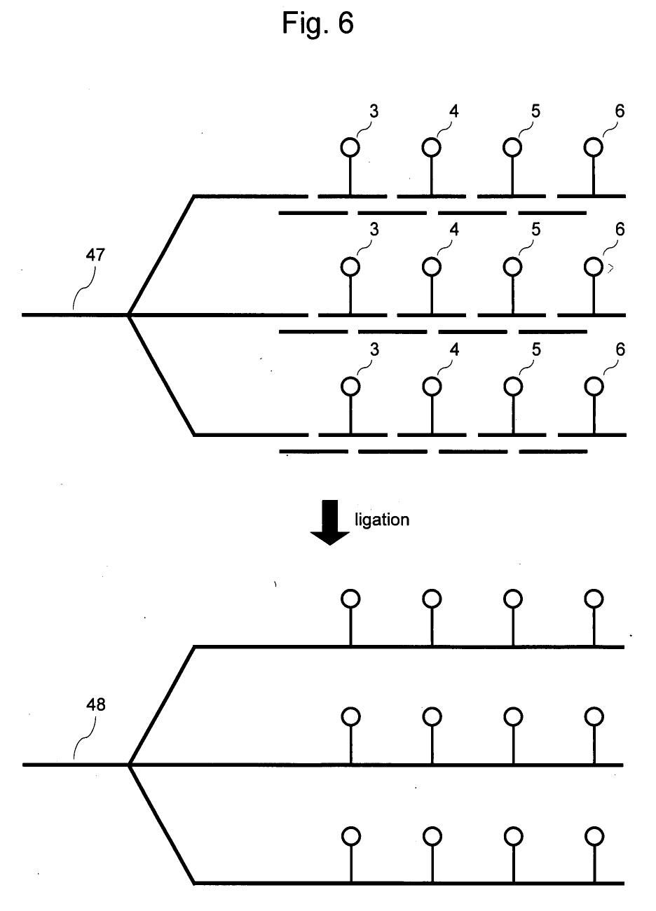

For a branched probe as well, oligonucleotides (complementary oligonucleotides) complementary to probes in branch portions can be prepared, followed by ligation to thereby obtain an oligonucleotide comprising plural fluorophores introduced into each of the branch portions. As shown in FIG. 6, a branched oligonucleotide (probe unit for analyte nucleic acid recognition) 47 hybridizing to an analyte molecule can be linked to oligonucleotides (labeled probe units) 3, 4, 5, and 6 each having a fluorophore labeled base that is located internal except at the 5′-end and the 3′-end, to thereby introduce plural fluorophores into each of the branch portions. This can control the number of labels while synthesizing a branched probe 48 comprising plural fluorophores introduced therein.

As described above, an oligonucleotide (probe unit for analyte nucleic acid recognition) having a sequence recognizing an analyte molecule sequence and fluorophore labeled oligonucleotides (labeled probe units) can be linked by ligation to thereby control the number of fluorophores while synthesizing a probe labeled with plural fluorophores. To detect another analyte molecule, only the sequence recognizing an analyte molecule may be changed, and the fluorophore labeled portions can be utilized universally, irrespective of analyte molecule types. Specifically, the present invention also provides a kit for synthesis of these fluorophore labeled probes that can be utilized universally. The kit of the present invention may be intended for particular analyte molecule analysis or may be provided in a form wherein a user prepares an oligonucleotide having a sequence recognizing an analyte molecule and can easily synthesize fluorophore labeled probes for various analyte molecules. Fluorophore labeled oligonucleotides (labeled probe units), fluorophores, and linkers for fluorophore introduction into the oligonucleotides as essential components in the kit are as described above. The present invention synthesizes a probe comprising plural fluorophores introduced into a linear oligonucleotide or branched oligonucleotide strand. In this probe, the fluorophore labels of bases are placed at a distance of eight or more bases.

The kit of the present invention may comprise other reagents necessary for probe synthesis, in addition to the fluorophore labeled oligonucleotides, fluorophores, and linkers for fluorophore introduction into the oligonucleotides as essential components. Examples of such reagents can include reagents such as enzymes, buffers that provide conditions suitable to enzyme reaction, and substrates necessary for enzyme reaction. Furthermore, the kit may provide reagents necessary for one reaction dispensed into reaction containers.

### EXAMPLES

Hereinafter, the present invention will be described more specifically with reference to Examples. However, the present invention is not intended to be limited to these Examples.

### Example 1

### Synthesis Method of Oligonucleotide Probe Having Plural Fluorophore Labels by Ligation

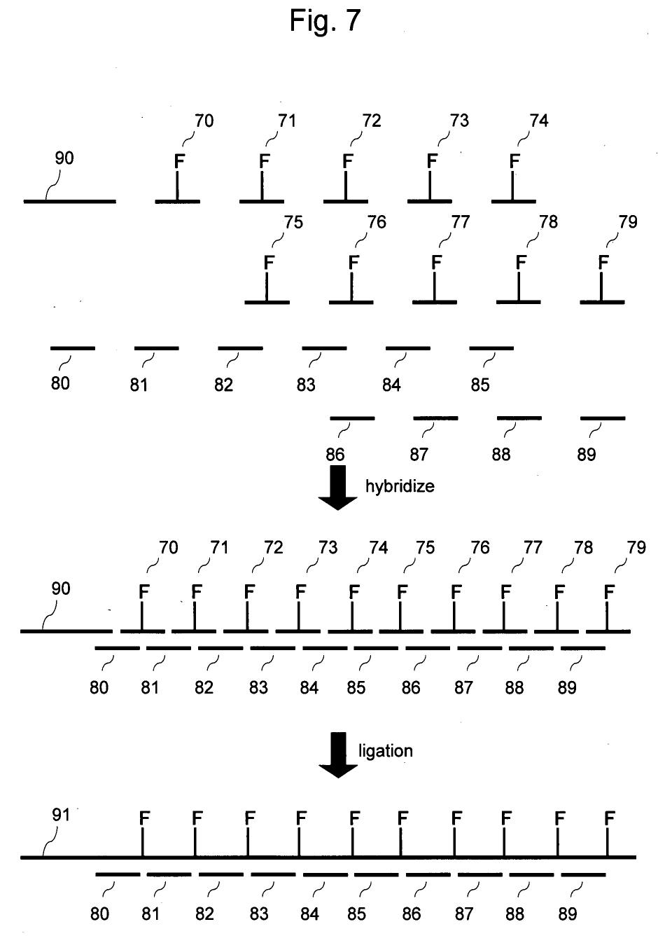

This Example shows the synthesis of an oligonucleotide probe having plural fluorophore labels by use of a method of the present invention. Ten kinds of oligonucleotides (labeled probe units) 70, 71, 72, 73, 74, 75, 76, 77, 78, and 79 (SEQ ID NOs: 1 to 10) were prepared, each of which comprised an 18-base sequence and had internal T (thymidine) at the 10th base position labeled with a fluorophore FITC. Ten kinds of oligonucleotides (complementary oligonucleotides) 80, 81, 82, 83, 84, 85, 86, 87, 88, and 89 (SEQ ID NOs: 11 to 20) were prepared which hybridized to the 10 kinds of oligonucleotides (labeled probe units) to form a sticky end. Moreover, an oligonucleotide (probe unit for analyte nucleic acid recognition) 90 (SEQ ID NO: 21) was prepared which hybridized to both an analyte molecule and the oligonucleotide 80 prepared in advance. Each oligonucleotide sequence was designed to form double-stranded DNA, as shown in FIG. 7.

The oligonucleotides were mixed at each amount of 50 pmol and subjected to ligation through reaction at 37° C. for 60 minutes by use of Invitrogen T4 ligase to thereby obtain a probe 91 comprising the 10 kinds of fluorophore labeled oligonucleotides and the oligonucleotide for analyte molecule recognition linked together. The fluorophore labeled base in the fluorophore labeled oligonucleotide of 18 bases in length is located at the 10th base position. Therefore, the fluorophore labeled oligonucleotide has 9 fluorophore unlabeled bases at the 5′-end and 8 fluorophore unlabeled bases at the 3′-end. Thus, the probe obtained after the ligation has the fluorophore labeled bases every 18 bases.

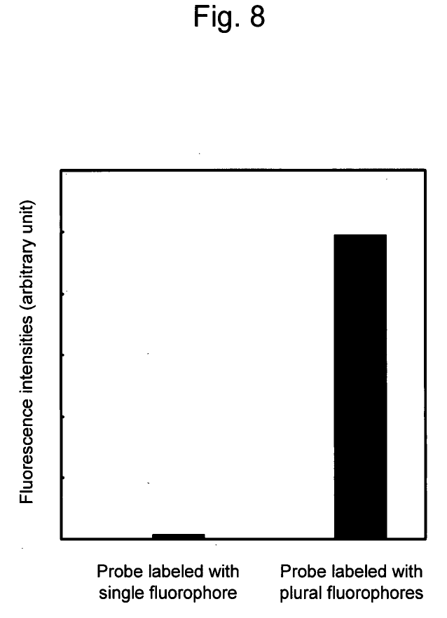

After the completion of reaction, the ligase was inactivated, and the fluorescence intensity of the obtained solution was measured. FIG. 8 shows fluorescence intensity measurement results of the fluorophore labeled oligonucleotide 70 before reaction and the probe 91 obtained by reaction. Since plural fluorophores were introduced into one probe molecule, fluorescence intensity per unit concentration could be confirmed to be increased by 10 or more times.

| | |
|---|---|
| FITC labeled oligonucleotide 70: | |
| 5′-aagtgacctttaaacatg-3′ | (SEQ ID NO: 1) |
| FITC labeled oligonucleotide 71: | |
| 5′-aagtgagctttaaaccag-3′ | (SEQ ID NO: 2) |
| FITC labeled oligonucleotide 72: | |
| 5′-aagtggtctttaaacgcg-3′ | (SEQ ID NO: 3) |
| FITC labeled oligonucleotide 73: | |
| 5′-aagtgtactttaaactgg-3′ | (SEQ ID NO: 4) |
| FITC labeled oligonucleotide 74: | |
| 5′-aagtgatctttaaacacg-3′ | (SEQ ID NO: 5) |
| FITC labeled oligonucleotide 75: | |
| 5′-acttgacctttccacatg-3′ | (SEQ ID NO: 6) |
| FITC labeled oligonucleotide 76: | |
| 5′-acttgagctttccaccag-3′ | (SEQ ID NO: 7) |
| FITC labeled oligonucleotide 77: | |
| 5′-acttggtctttccacgcg-3′ | (SEQ ID NO: 8) |
| FITC labeled oligonucleotide 78: | |
| 5′-acttgtactttccactgg-3′ | (SEQ ID NO: 9) |
| FITC labeled oligonucleotide 79: | |
| 5′-acttgatctttccacacg-3′ | (SEQ ID NO: 10) |
| oligonucleotide 80: | |
| 5′-ggtcacttgcacattt-3′ | (SEQ ID NO: 11) |
| oligonucleotide 81: | |
| 5′-gctcacttcatgtttaa-3′ | (SEQ ID NO: 12) |
| oligonucleotide 82: | |
| 5′-gaccacttctggtttaa-3′ | (SEQ ID NO: 13) |
| oligonucleotide 83: | |
| 5′-gtacacttcgcgtttaa-3′ | (SEQ ID NO: 14) |
| oligonucleotide 84: | |
| 5′-gatcacttccagtttaa-3′ | (SEQ ID NO: 15) |
| oligonucleotide 85: | |
| 5′-ggtcaagtcgtgtttaa-3′ | (SEQ ID NO: 16) |
| oligonucleotide 86: | |
| 5′-gctcaagtcatgtggaa-3′ | (SEQ ID NO: 17) |
| oligonucleotide 87: | |
| 5′-gaccaagtctggtggaa-3′ | (SEQ ID NO: 18) |
| oligonucleotide 88: | |
| 5′-gtacaagtcgcgtggaa-3′ | (SEQ ID NO: 19) |
| oligonucleotide 89: | |
| 5′-gatcaagtccagtggaa-3′ | (SEQ ID NO: 20) |
| oligonucleotide 90: | |
| 5′- | (SEQ ID NO: 21) |
| ctagccgagtagtgttgggttttaaatgtgc-3′ | |

### Example 2

### Difference in Ligation Efficiency Depending on Difference in Position of Fluorophore Label

Fluorophore labeled oligonucleotides were linked together by ligation in the same way as in Example 1 to thereby synthesize a probe labeled with plural fluorophores.

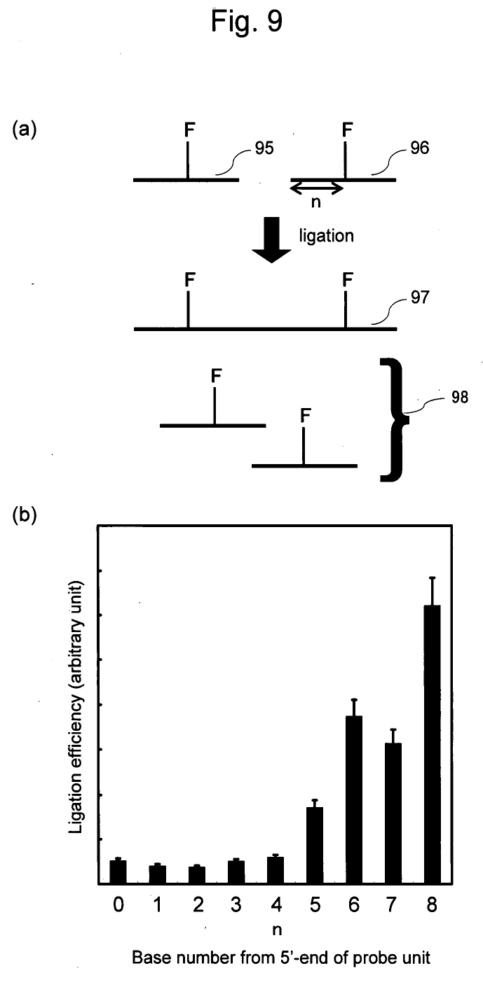

Of two fluorophore labeled oligonucleotides (labeled probe units) 95 and 96 to be linked, the oligonucleotide 96 was subjected to ligation using varying positions of its fluorophore label, as shown in FIG. 9(A). After the electrophoresis of the reaction products, the amounts of the ligation product 97 and the unreacted product 98 were measured with a densitometer to calculate ligation efficiency. As a result, when the fluorophore label of the oligonucleotide 96 was located at the 5′-end to at the fourth base position, ligation efficiency was low, demonstrating that these positions are not suitable for probe synthesis, as shown in FIG. 9(B). The same tendency was also observed at the 3′-end. This suggested that the position of a fluorophore label within the fourth bases from both the ends inhibits enzyme function and thereby reduces ligation efficiency. Therefore, the introduction position of a fluorophore label was set to a position five or more bases internal from the 5′-end and 3′-end of an oligonucleotide in subsequent Examples.

### Example 3

### Probe Synthesis Method Comprising Gap Filling Step Using DNA Polymerase Before Ligation

This Example shows probe synthesis by use of the method of the present invention, tcomprising the hybridization of oligonucleotides having plural fluorophore labels to complementary oligonucleotides, elongation reaction with DNA polymerase, and subsequent ligation.

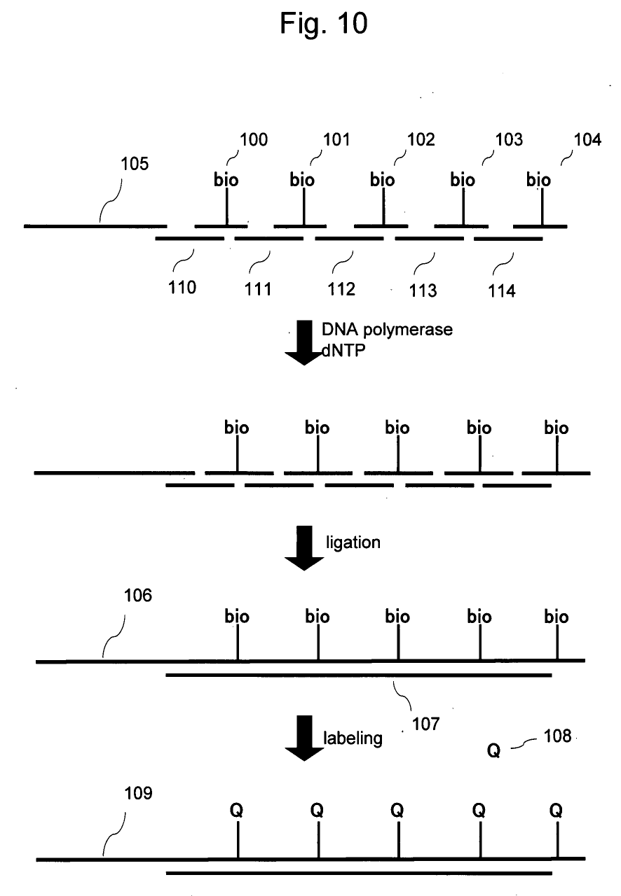

Five kinds of oligonucleotides (labeled probe units) 100, 101, 102, 103, and 104 (SEQ ID NOs: 22 to 26) were prepared, each of which comprised a 16-base sequence and had internal T (thymidine) at the 9th base position labeled with biotin. Five kinds of oligonucleotides (complementary oligonucleotides) 110, 111, 112, 113, and 114 (SEQ ID NOs: 27 to 31) were prepared which hybridized to the five kinds of oligonucleotides (labeled probe units) prepared. Moreover, an oligonucleotide (probe unit for analyte nucleic acid recognition) 105 (SEQ ID NO: 32) was prepared which recognized and hybridized to an analyte molecule and the oligonucleotide 110. Each oligonucleotide sequence was designed to form double-stranded DNA but to produce an at least one-base or more gap, as shown in FIG. 10. The fluorophore labeled base in the fluorophore labeled oligonucleotide of 16 bases in length is located at the ninth base position. Therefore, the fluorophore labeled oligonucleotide has eight fluorophore unlabeled bases at the 5′-end and seven fluorophore unlabeled bases at the 3′-end. Thus, the probe obtained after the filling of two-base gaps and ligation has the fluorophore labeled bases every 18 bases.

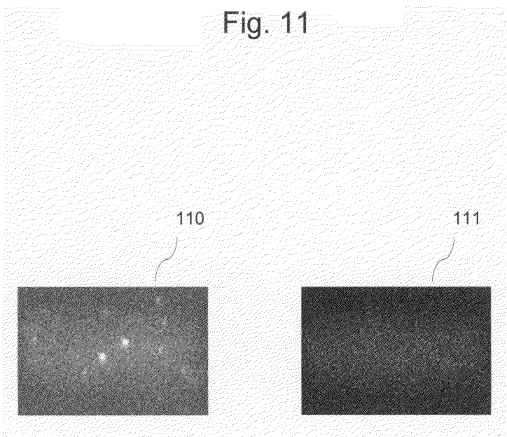

The oligonucleotides were mixed at each amount of 50 pmol and elongated through reaction at 37° C. for 10 minutes in a buffer solution containing dNTP and DNA polymerase to thereby cause reaction for the filling of two-base gaps. After this elongation reaction, ligation was performed to thereby obtain a probe 106 comprising the biotin labeled oligonucleotides and the oligonucleotide for analyte molecule recognition linked together. In this procedure, complementary strands of the probe were also linked by the ligation to form a complementary strand 107. The probe 106 and quantum dots 108 labeled with streptavidin were mixed to thereby obtain a probe 109 labeled with plural quantum dots. FIG. 11 shows a result of measurement of the obtained probe with a fluorescence microscope. The probes that emitted fluorescence were observed in an image 110 showing the measurement of the probe 109, whereas no distinct fluorescent image was observed in an image 111 showing the measurement of a probe comprising one quantum dot introduced therein, demonstrating that the present invention can easily increase the florescence intensity of a probe.

| | |
|---|---|
| biotin labeled oligonucleotide | |
| 100: | |
| 5′- gatgagctttggacca-3′ | (SEQ ID NO: 22) |
| biotin labeled oligonucleotide | |
| 101: | |
| 5′- gatggtctttggacgc-3′ | (SEQ ID NO: 23) |
| biotin labeled oligonucleotide | |
| 102: | |
| 5′- gatgtactttggactg-3′ | (SEQ ID NO: 24) |
| biotin labeled oligonucleotide | |
| 103: | |
| 5′- gatgatctttggacac-3′ | (SEQ ID NO: 25) |
| biotin labeled oligonucleotide | |
| 104: | |
| 5′- tctgacctttttacat-3′ | (SEQ ID NO: 26) |
| oligonucleotide 110: | |
| 5′- gctcatctcatgtcca-3′ | (SEQ ID NO: 27) |
| oligonucleotide 111: | |
| 5′- gaccatctctggtcca-3′ | (SEQ ID NO: 28) |
| oligonucleotide 112: | |
| 5′- gtacatctcgcgtcca-3′ | (SEQ ID NO: 29) |
| oligonucleotide 113: | |
| 5′- gatcatctccagtcca-3′ | (SEQ ID NO: 30) |
| oligonucleotide 114: | |
| 5′- ggtcagatcgtgtcca-3′ | (SEQ ID NO: 31) |
| oligonucleotide 105: | |
| 5′- | |
| ctagccgagtagtgttgggttttatggacat-3′ | (SEQ ID NO: 32) |

The present invention can conveniently and inexpensively synthesize a probe labeled with plural fluorophores by linking oligonucleotides each having a fluorophore labeled internal base by ligation. This can achieve improvement in the performance of probes used for various fluorescence detections and in detection limit and allows for single-molecule measurement with sufficient sensitivity. Thus, the present invention is useful in wide fields from basic research to clinical fields, such as the quantification of trace amounts of nucleic acids and disease diagnosis using this quantification.

### [Free Text of Sequence Listing]

SEQ ID NO: 1—Description of artificial sequence: FITC labeled probe unit
SEQ ID NO: 2—Description of artificial sequence: FITC labeled probe unit
SEQ ID NO: 3—Description of artificial sequence: FITC labeled probe unit
SEQ ID NO: 4—Description of artificial sequence: FITC labeled probe unit
SEQ ID NO: 5—Description of artificial sequence: FITC labeled probe unit
SEQ ID NO: 6—Description of artificial sequence: FITC labeled probe unit
SEQ ID NO: 7—Description of artificial sequence: FITC labeled probe unit
SEQ ID NO: 8—Description of artificial sequence: FITC labeled probe unit
SEQ ID NO: 9—Description of artificial sequence: FITC labeled probe unit
SEQ ID NO: 10—Description of artificial sequence: FITC labeled probe unit
SEQ ID NO: 11—Description of artificial sequence: oligonucleotide complementary to probe unit
SEQ ID NO: 12—Description of artificial sequence: oligonucleotide complementary to probe unit
SEQ ID NO: 13—Description of artificial sequence: oligonucleotide complementary to probe unit
SEQ ID NO: 14—Description of artificial sequence: oligonucleotide complementary to probe unit
SEQ ID NO: 15—Description of artificial sequence: oligonucleotide complementary to probe unit
SEQ ID NO: 16—Description of artificial sequence: oligonucleotide complementary to probe unit
SEQ ID NO: 17—Description of artificial sequence: oligonucleotide complementary to probe unit
SEQ ID NO: 18—Description of artificial sequence: oligonucleotide complementary to probe unit
SEQ ID NO: 19—Description of artificial sequence: oligonucleotide complementary to probe unit
SEQ ID NO: 20—Description of artificial sequence: oligonucleotide complementary to probe unit
SEQ ID NO: 21—Description of artificial sequence: probe unit hybridizing to analyte molecule
SEQ ID NO: 22—Description of artificial sequence: biotin labeled probe unit
SEQ ID NO: 23—Description of artificial sequence: biotin labeled probe unit
SEQ ID NO: 24—Description of artificial sequence: biotin labeled probe unit
SEQ ID NO: 25—Description of artificial sequence: biotin labeled probe unit
SEQ ID NO: 26—Description of artificial sequence: biotin labeled probe unit
SEQ ID NO: 27—Description of artificial sequence: oligonucleotide complementary to probe unit
SEQ ID NO: 28—Description of artificial sequence: oligonucleotide complementary to probe unit
SEQ ID NO: 29—Description of artificial sequence: oligonucleotide complementary to probe unit
SEQ ID NO: 30—Description of artificial sequence: oligonucleotide complementary to probe unit
SEQ ID NO: 31—Description of artificial sequence: oligonucleotide complementary to probe unit
SEQ ID NO: 32—Description of artificial sequence: probe unit hybridizing to analyte molecule

What is claimed is:

1. An analytical probe production method comprising:
a first step wherein a probe unit for analyte nucleic acid recognition having a base sequence specific to an analyte nucleic acid and plural labeled probe units having mutually different base sequences and each being labeled with a fluorophore are hybridized to plural complementary oligonucleotides each having a sequence complementary to a portion of the neighborhood of the 5′-end of one of the probe units and to a portion of the neighborhood of the 3′-end of one of the other probe units to thereby obtain a hybridized probe unit comprising the probe unit for analyte nucleic acid recognition, the plural labeled probe units, and the plural complementary oligonucleotides; and
a second step wherein the adjacent probe units in the hybridized probe unit are bonded by ligation to thereby obtain an analytical probe labeled with plural fluorophores.

2. The analytical probe production method according to claim 1, further comprising a step wherein DNA polymerase and substrate dNTP are allowed to act on the hybridized probe unit before the ligation to thereby fill the gaps between the probe units.

3. The analytical probe production method according to claim 1, wherein the fluorophore is bound with a base located five or more bases internal from the 5′-end and 3′-end of the labeled probe unit.

4. The analytical probe production method according to claim 1, wherein the fluorophore is bound with a base located substantially at the center of the labeled probe unit.

5. The analytical probe production method according to claim 1, wherein the labeled probe unit has a length between 9 nucleotides and 30 nucleotides inclusive.

6. The analytical probe production method according to claim 1, wherein the plural labeled probe units are labeled with the fluorophores that are mutually identical.

7. The analytical probe production method according to claim 1, wherein the plural labeled probe units are labeled with the fluorophores that are mutually different.

8. An analytical probe production method comprising:
a first step wherein a probe unit for analyte nucleic acid recognition having a base sequence specific to an analyte nucleic acid and plural probe units to be labeled having mutually different base sequences are hybridized to plural complementary oligonucleotides each having a sequence complementary to a portion of the neighborhood of the 5′-end of one of the probe units and to a portion of the neighborhood of the 3′-end of one of the other probe units to thereby obtain a hybridized probe unit comprising the probe unit for analyte nucleic acid recognition, the plural probe units to be labeled, and the plural complementary oligonucleotides;
a second step wherein the adjacent probe units in the hybridized probe unit are bonded by ligation; and
a third step wherein a fluorophore is introduced into each of the plural probe units to be labeled to thereby obtain an analytical probe labeled with plural fluorophores.

9. The analytical probe production method according to claim 8, wherein the method further comprises a step wherein DNA polymerase and substrate dNTP are allowed to act on the hybridized probe unit before the ligation to thereby fill the gaps between the probe units.

10. The analytical probe production method according to claim 8, wherein the fluorophore is bound with a base located five or more bases internal from the 5′-end and 3′-end of the probe unit to be labeled.

11. The analytical probe production method according to claim 8, wherein the fluorophore is bound with a base located substantially at the center of the probe unit to be labeled.

12. The analytical probe production method according to claim 8, wherein the probe unit to be labeled has a length between 9 nucleotides and 30 nucleotides inclusive.

13. The analytical probe production method according to claim 8, wherein the fluorophores that are mutually identical are introduced into the plural probe units to be labeled.

14. The analytical probe production method according to claim 8, wherein the fluorophores that are mutually different are introduced into the plural probe units to be labeled.

15. The analytical probe production method according to claim 1, wherein the probe unit for analyte nucleic acid recognition is a branched probe having branches.

16. A kit for analytical probe production comprising: plural probe units having mutually different base sequences; and plural complementary oligonucleotides each having a sequence complementary to a portion of the neighborhood of the 5′-end of one of the probe units and to a portion of the neighborhood of the 3′-end of one of the other probe units.

17. The kit for analytical probe production according to claim 16, wherein one of the plural probe units is a probe unit for analyte nucleic acid recognition having a base sequence specific to an analyte nucleic acid.

18. The kit for analytical probe production according to claim 17, wherein the plural probe units other than the probe unit for analyte nucleic acid recognition are labeled probe units each labeled with a fluorophore.

19. The kit for analytical probe production according to claim 18, wherein the labeled probe unit has a length between 9 nucleotides and 30 nucleotides inclusive, and the fluorophore is bound with a base located five or more bases internal from the 5′-end and 3′-end of the labeled probe unit.

20. An analytical probe comprising: a probe unit for analyte nucleic acid recognition having a base sequence specific to an analyte nucleic acid; and plural tandemly ligated labeled probe units having mutually different base sequences and each comprising a fluorophore labeled internal base, wherein the fluorophore-labeled internal bases are placed at a distance of eight or more bases.
